(19) United States
(12) Patent Application Publication
(10) Pub. No.: US 2015/0272767 A1
(43) Pub. Date: Oct. 1, 2015

(54) ATHLETIC BRACE WITH INNER GRIPPING LAYER

(71) Applicant: Joshua Field, Swampscott, MA (US)

(72) Inventor: Joshua Field, Swampscott, MA (US)

(21) Appl. No.: 14/675,077

(22) Filed: Mar. 31, 2015

Related U.S. Application Data
(60) Provisional application No. 61/972,994, filed on Mar. 31, 2014.

(51) Int. Cl. A61F 5/01

(52) U.S. Cl. CPC A61F 5/0123

(57) ABSTRACT

An athletic brace is provided. The athletic brace has a fabric layer and a gripping layer. The fabric layer includes an inner surface and an outer surface and is shaped to secure to a portion of the user's body so that the inner surface of the fabric layer is facing the skin. The gripping layer is attached to the inner surface, and thereby is in direct contact with the skin. The gripping layer provides additional support to an injured area and promotes blood flow.

### CROSS-REFERENCE TO RELATED APPLICATION

This application claims the benefit of priority of U.S. provisional application No. 61/972,994, filed Mar. 31, 2014, the contents of which are herein incorporated by /reference.

### BACKGROUND OF THE INVENTION

The present invention relates to an athletic brace and, more particularly, to an athletic brace with an inner gripping layer.

Elastic therapeutic tape or kinesio tape is an elastic cotton strip with an acrylic adhesive that is used with the intent of treating pain and disability from athletic injuries and a variety of other physical disorders. Currently, kinesio tape is difficult to apply to your body without the help of others. Further, perspiration from the user's skin interacts with the adhesive of the tape, causing the tape to peel away.

As can be seen, there is a need for a brace that has a gripping effect similar to elastic therapeutic tape.

### SUMMARY OF THE INVENTION

In one aspect of the present invention, an athletic brace comprises: a fabric layer comprising an inner surface and an outer surface, wherein the fabric layer is shaped to secure to a portion of a human body so that the inner surface is facing the portion; and a gripping layer attached to the inner surface at least at a portion centered in between the upper edge and the lower edge of the fabric layer, wherein the gripping layer comprises an elastomer.

In another aspect of the present invention, an athletic brace comprises: a fabric layer comprising an inner surface and an outer surface, wherein the fabric layer is shaped to secure to a portion of a human body so that the inner surface is facing the portion; and a gripping layer attached to the inner surface and positioned to cover an injured area of the portion of the human body, wherein the gripping layer comprises an elastomer.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is a cutaway perspective view of an embodiment of the present invention;

FIG. 3 is a detail perspective view of an embodiment of the present invention;

FIG. 4 is a section view of an embodiment of the present invention;

FIG. 5 is a section view of an embodiment of the present invention;

FIG. 6 is a perspective view of an embodiment of the present invention;

FIG. 7 is a cutaway perspective view of an embodiment of the present invention;

FIG. 8 is a cutaway perspective view of an embodiment of the present invention; and

FIG. 9 is a cutaway perspective view of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The preset invention includes an athletic brace with a lining of silicone, or similar material applied to the inside of the brace that provides a grasping affect to the skin. The brace of the present invention is easy for one person to apply and it relies on compression to stay in place as opposed to an adhesive. The kinetic panels grip and massage the skin helping to promote blood flow to the targeted area. Through a combined effect of compression and the grasping of the skin, the brace helps facilitate the natural healing process of soft tissue injuries.

Referring to FIGS. 1 through 9, the present invention includes an athletic brace. The athletic brace has a fabric layer 10 and a gripping layer 12. The fabric layer 10 includes an inner surface and an outer surface and is shaped to secure to a portion 16 of the user's body 14 so that the inner surface of the fabric layer 10 is facing the skin. The gripping layer 12 is attached to the inner surface, and thereby is in direct contact with the skin. In certain embodiments, a logo or indicator may be on the outer surface, indicating where the gripping layer 12 is located on the inner surface. At least a portion of the gripping layer 12 may be centered in between an upper edge and a lower edge of the athletic brace in order to cover an injured area of the user's body 14. The gripping layer 12 provides additional support to an injured area and promotes blood flow.

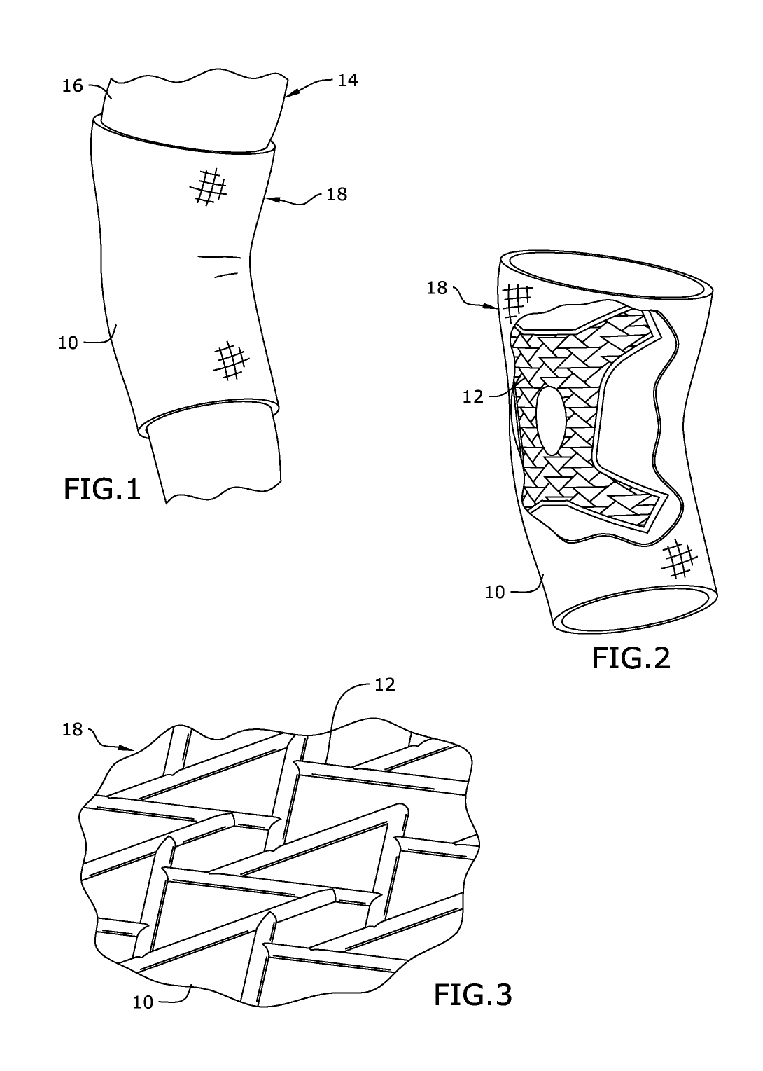

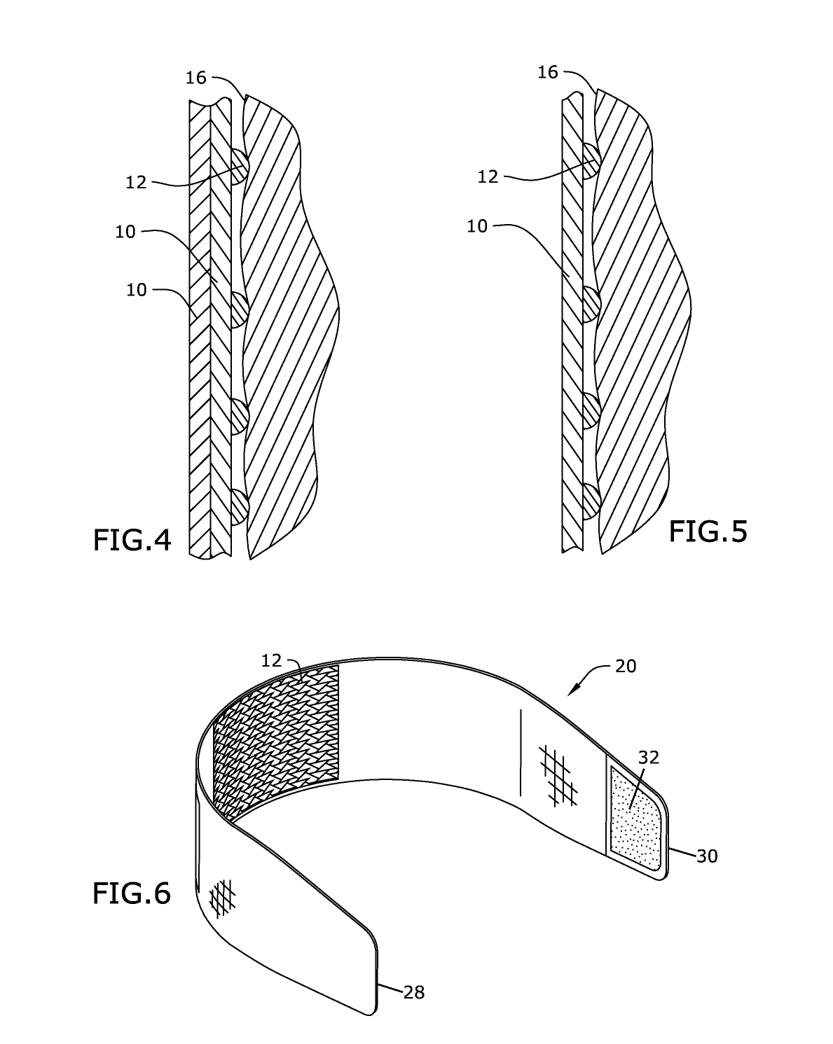

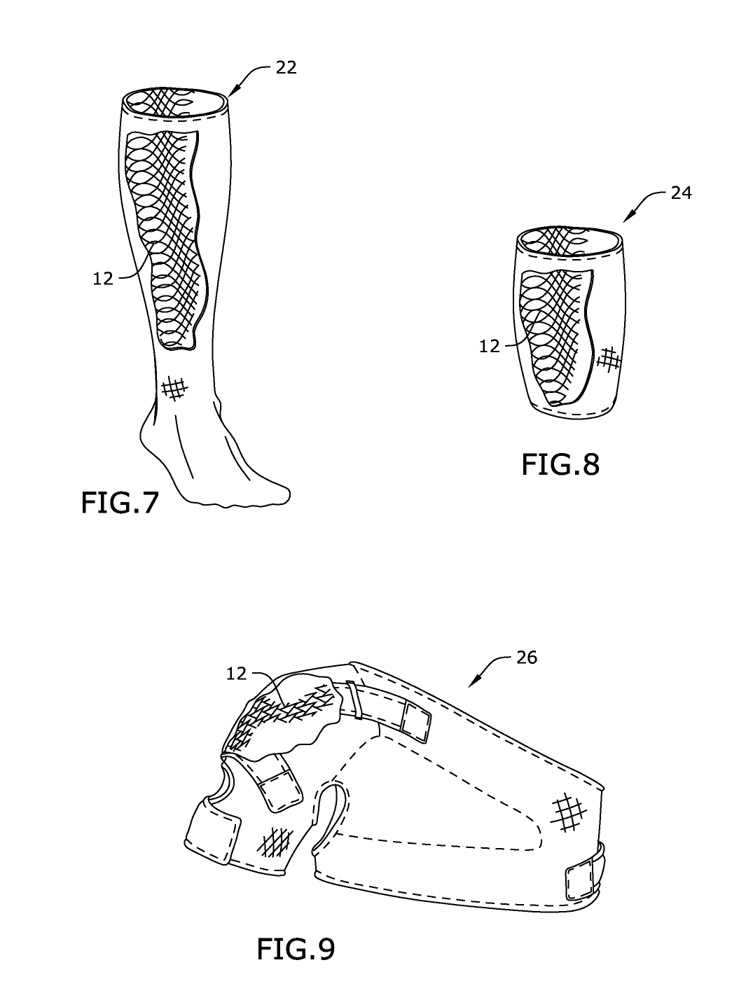

The gripping layer 12 of the present invention is a polymer. In certain embodiments, the polymer may be a rubber polymer, such as an elastomer. For example, the polymer may be a silicone rubber. The gripping layer 12 may be adhered to the inner surface or woven into the inner surface of the fabric layer 10. As illustrated in FIG. 4, the fabric layer 10 may have multiple layers and in such embodiments the gripping layer 12 may be attached to the inner surface of the inner layer. The gripping layer 12 may be in the form of a plurality of filaments, thereby adding additional grip. The filaments may protrude from the inner surface and into the skin. In certain embodiments, the plurality of filaments may be intersecting. As illustrated in FIGS. 2 and 3, the plurality of intersecting filaments may be straight lines and form a triangular shape. However, as illustrated in FIGS. 7 and 8, the plurality of intersecting filaments may be curved and form intersecting waves.

As illustrated in the Figures, the athletic brace may be in a tubular shape to fit over a user's knee, arm, shoulder and the like. In certain embodiments, the athletic brace may lay flat with a first end 28 and a second end 30. The athletic brace may be wrapped around an appendage of the user's body 14 and the first end 28 and second end 30 may releasably attach to one another by a connector 32. For example, the connector 32 may include, but is not limited to, clips, buckles, straps, hook and loop fasteners, and the like. Therefore, the athletic brace may be tightened or loosened around the user's body 14.

The athletic brace may be a knee brace 18 so that the gripping layer 12 is positioned to secure over a knee. The athletic brace may be a back brace 20 so that the gripping layer 12 is positioned to secure over a back. The athletic brace may be a shoulder brace 26 so that the gripping layer is positioned to secure over a shoulder. The athletic brace may be a sock 22 so that the gripping layer 12 is positioned to secure over at least one of a calf and an ankle. The athletic brace may be a calf brace 24 so that the gripping layer 12 is positioned to secure over a calf. The athletic brace may be used to cover other portions of the body, such as but not limited to, the elbow, thigh, ankle, foot, wrist, forearm, and the like.

A method of using the present invention may include the following. Position the support by sliding it on, or by wrapping it around the desired location and attaching the connectors. The kinetic silicone panels are aligned in the correct location using the logos or indicators on the exterior side of the fabric material for reference to ensure the support is positioned correctly.

It should be understood, of course, that the foregoing relates to exemplary embodiments of the invention and that modifications may be made without departing from the spirit and scope of the invention as set forth in the following claims.

What is claimed is:

1. An athletic brace comprising:
a fabric layer comprising an inner surface, an outer surface, an upper edge and a lower edge, wherein the fabric layer is shaped to secure to a portion of a human body so that the inner surface is facing the portion; and
a gripping layer attached to the inner surface at least at a portion centered in between the upper edge and the lower edge of the fabric layer, wherein the gripping layer comprises an elastomer.

2. The athletic brace of claim 1, wherein the gripping layer comprises a plurality of intersecting polymer filaments.

3. The athletic brace of claim 2, wherein the plurality of intersecting polymer filaments are straight.

4. The athletic brace of claim 2, wherein the plurality of intersecting polymer filaments form a triangular pattern.

5. The athletic brace of claim 1, wherein the elastomer is a silicone rubber.

6. The athletic brace of claim 1, wherein the athletic brace is a knee brace, wherein the gripping layer is positioned to secure over a knee.

7. The athletic brace of claim 1, wherein the athletic brace is a back brace, wherein the gripping layer is positioned to secure over a back.

8. The athletic brace of claim 1, wherein the athletic brace is a shoulder brace, wherein the gripping layer is positioned to secure over a shoulder.

9. The athletic brace of claim 1, wherein the athletic brace is a sock, wherein the gripping layer is positioned to secure over at least one of a calf and an ankle.

10. The athletic brace of claim 1, wherein the athletic brace is a calf brace, wherein the gripping layer is positioned to secure over a calf.

11. The athletic brace of claim 1, wherein the fabric is formed in a tubular shape.

12. The athletic brace of claim 11, wherein the tubular shape comprises a first end and a second end releasably attachable to one another by a connector.

13. The athletic brace of claim 12, wherein the connector is a hook and loop fastener.

14. An athletic brace comprising:
a fabric layer comprising an inner surface, an outer surface, an upper edge and a lower edge, wherein the fabric layer is shaped to secure to a portion of a human body so that the inner surface is facing the portion; and
a gripping layer attached to the inner surface and positioned to cover an injured area of the portion of the human body, wherein the gripping layer comprises an elastomer.

15. The athletic brace of claim 14, wherein the gripping layer comprises a plurality of intersecting polymer filaments.

16. The athletic brace of claim 14, wherein the athletic brace is a calf brace, wherein the gripping layer is positioned to secure over a calf.

17. The athletic brace of claim 14, wherein the fabric is formed in a tubular shape.

18. The athletic brace of claim 17, wherein the tubular shape comprises a first end and a second end releasably attachable to one another by a connector.
